(19) United States
(12) Patent Application Publication
(10) Pub. No.: US 2010/0087398 A1
(43) Pub. Date: Apr. 8, 2010

(54) DIHYDROPYRIDINE DERIVATIVE FOR TREATING CANCER OR A PRE-CANCEROUS CONDITION AND OTHER CONDITIONS

(75) Inventor: Andrew J. Krouse, Charlottesville, VA (US)

Correspondence Address: SCULLY, SCOTT, MURPHY & PRESSER, P.C., 400 GARDEN CITY PLAZA, SUITE 300, GARDEN CITY, NY, 11530, US

(73) Assignee: Tau Therapeutics LLC, Charlottesville, VA (US)

(21) Appl. No.: 12/598,499

(22) PCT Filed: May 2, 2008
(86) PCT No.: PCT/US08/05731

§ 371 Date: Nov. 2, 2009

Related U.S. Application Data
(60) Provisional application No. 60/927,301, filed on May 2, 2007.

(51) Int. Cl. A61K 31/675; A61P 35/04

(52) U.S. Cl. 514/89

(57) ABSTRACT

The use of dihydropyridine-5-phosphonic acid cyclic propylene ester derivatives of formula (I), a prodrug thereof, or a pharmaceutically acceptable salt of said compound or prodrug in the treatment of cancers, pre-cancerous conditions and other conditions is disclosed, wherein each of R₁-R₈ are the same or different, are hydrogen or C₁-C₆ alkyl; one of X₁ and X₂ is nitro while the other is hydrogen; each of Y₁ and Y₂ may be the same or different, is phenyl which may be substituted by chlorine, fluorine or alkoxy; and m and n are integers from 0-4, a prodrug thereof, or a pharmaceutically acceptable salt thereof.

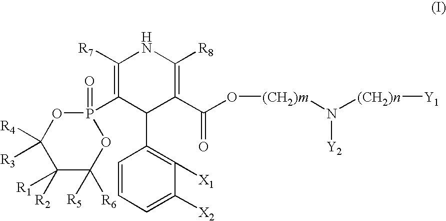

### BACKGROUND OF THE INVENTION

Cancer is a significant health problem throughout the world. In fact, cancer is the number two leading cause of death in America. Although advances have been made in detection and therapy of cancer, no vaccine or other universally successful method for prevention or treatment is currently available. Conventional cancer therapies focus on cytotoxic treatments, such as chemotherapy and radiation. However, cytotoxic treatments are indiscriminately detrimental to both cancerous cells and normally dividing cells, and thereby tend to cause severe side effects or even secondary cancers. One of the most common toxic manifestations of cytotoxic agents is bone marrow suppression which can lead to immune suppression and hematopoietic dysfunctions. Moreover, cytotoxic treatments induce drug-resistant cancer cells producing tumors that are increasingly difficult to eradicate. Another conventional cancer therapy is surgery. The challenge facing surgery is that it needs to be 100% effective because even a single remaining cancer cell can regenerate the tumor. Therefore, the current therapies, which are generally based on a combination of chemotherapy or surgery and radiation, continue to prove inadequate in the treatment of cancer.

In recent years, promising progress has been made in the development of cytostatic chemotherapy. The hallmark of cancer cells is the uncontrolled and dysregulated proliferation that erode the anatomic and physiologic integrity of the body and ultimately lead to the death of the patient. Cytostatic chemotherapy aims to reduce the proliferative rate of cancer to that of healthy tissues by using cytostatic agents that can retard cellular activity and multiplication of cancer cells. If the proliferation of cancer cells is effectively controlled, cancer will no longer be a terminal disease, and instead will become analogous to the treatment for other chronic diseases. Moreover, cytostatic chemotherapy can minimize the collateral damage to normal tissues caused by conventional cytotoxic drugs.

One type of cytostatic agents are antiangiogenic agents, a.k.a. angiogenic inhibitors. In experimental models, antiangiogenic agents were shown to starve the cancer cells by inhibiting the development of blood vessels that are essential for nourishing tumor growth and maintenance. However, antiangiogenic therapy, while promising, has serious limitations. The currently available antiangiogenic drugs are very expensive and must be administered by intravenous injection requiring a patient to regularly visit a medical clinic. Another disadvantage of antiangiogenic therapy is that it only targets a single component of the cancer's infrastructure. Recent clinical studies indicate that cancer cells can evolve to circumvent this single point blockade. Therefore, the need for developing new cytostatic agents for treating cancer is evident.

It is known that cellular activation and proliferation is regulated through control of calcium entry into the cells. The calcium influx in certain cells is regulated by electrical activity at the plasma membrane and these cells are called “electrically excitable”. The electrically excitable cells are exemplified by neurons and muscle cells. The calcium channels in these cells are termed “voltage gated” (VG) because they are regulated primarily by the change in voltage across the plasma membrane. All other cells, including lymphocytes and epithelial cells, lack the type of electrical activity occurring in electrically excitable cells and so are named “electrically non-excitable”. The calcium channels in these types of cell are also referred to as VG channels by researchers although calcium entry in these cells is conventionally believed to be conducted by non-voltage gated channels.

Knowledge about voltage gated calcium channels in electrically excitable cells has been exploited profitably. Pharmacological modulation of these channels' function is tremendously important in the practice of medicine; for example, calcium channel blockers are in widespread use in the treatment of neurological diseases (e.g. epilepsy) and cardiovascular diseases (e.g. hypertension and angina pectoris).

The majority of cancers arise from cell types considered electrically non-excitable. The entry of calcium ion into the cell at specific times in the cell cycle is known as essential for the proliferation of cancer cells. Thus, inhibitors of calcium entry in electrically non-excitable cells may be used for treating cancers as cytostatic agents. Recently issued U.S. Pat. No. 6,413,967 discloses methods for screening for VG-selective inhibitors and novel VG-selective inhibitors that can block calcium entry into electrically non-excitable cells. Therefore, it is evident that further investigation of calcium entry inhibitors, selective to electrically non-excitable cells, has significant clinical importance in cancer therapy.

Dihydropyridine derivatives have been used for the treatment of heart disease, circulatory disorders and hypertention since the 1980's (U.S. Pat. No. 4,535,073) and various dihydropyridine-5-phosphonic acid cyclic propylene ester has been synthesized (U.S. Pat. No. 4,885,284). For example, Efonidipine, a T-channel blocker, is known for treating pain. However, Efonidipine has not been employed to treat cancer, precancerous conditions and certain other conditions such as epilepsy, autism, diabetic nephropathy, diabetic neuropathy, age adjusted macular degeneration, and scars, burns and keloids.

The present invention, for the first time, provides a method of treating cancer or pre-cancerous conditions with dihydropyridine derivatives. The present invention further provides for a method of treating epilepsy, autism, diabetic nephropathy, diabetic neuropathy, age adjusted macular degeneration, and scars, burns and keloids.

### SUMMARY OF THE INVENTION

The present invention provides a method for the treatment of cancer or a pre-cancerous condition in a mammal, which comprises administering to the mammal a therapeutically effective amount of a compound of formula (I):

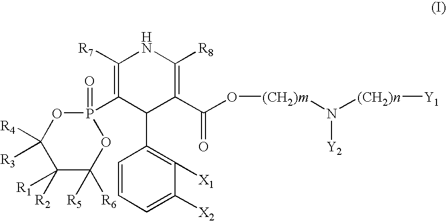

wherein each of R₁-R₈ are the same or different, are hydrogen or C₁-C₆ alkyl; one of X₁ and X₂ is nitro while the other is hydrogen; each of Y₁ and Y₂ may be the same or different, is phenyl which may be substituted by chlorine, fluorine or alkoxy; and m and n are integers from 0-4, a prodrug thereof, or a pharmaceutically acceptable salt thereof.

The present invention further provides a method for the treatment of epilepsy, autism, diabetic nephropathy, diabetic neuropathy, age adjusted macular degeneration, and scars, burns and keloids in a mammal, which comprises administering to the mammal a therapeutically effective amount of a compound of formula (I) as defined above, a prodrug thereof, or a pharmaceutically acceptable salt thereof.

In another aspect, the present invention provides pharmaceutical compositions comprising a therapeutically effective amount of a compound of formula (I), a prodrug of said compound or a pharmaceutically acceptable salt of said compound; and a pharmaceutically acceptable carrier, vehicle or diluent.

The present invention also provides a method for the treatment of cancer or pre-cancerous conditions in a mammal, which comprises administering to the mammal a therapeutically effective amount of a compound of formula (I), a prodrug thereof, or a pharmaceutically acceptable salt of said compound in combination with one or more antineoplastic agents.

The present invention further provides pharmaceutical combination compositions comprising a therapeutically effective amount of a combination of a compound of formula (I), a prodrug thereof, or a pharmaceutically acceptable salt of said compound; and one or more antineoplastic agent(s).

### DETAILED DESCRIPTION OF THE INVENTION

The present invention provides a method for the treatment of cancer or a pre-cancerous condition in a mammal, as well as for the treatment of epilepsy, autism, diabetic nephropathy, diabetic neuropathy, age adjusted macular degeneration, and scars, burns and keloids in a mammal, which comprises administering to the mammal a therapeutically effective amount of dihydropyridine-5-phosphonic acid cyclic propylene ester derivative, a prodrug thereof, or a pharmaceutically acceptable salt of said derivative or prodrug.

The dihydropyridine-5-phosphonic acid cyclic propylene ester derivative is preferably a compound of formula (I):

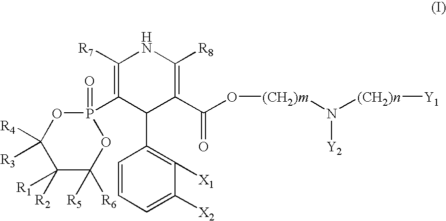

wherein each of R₁-R₈ are the same or different, are hydrogen or C₁-C₆ alkyl; one of X₁ and X₂ is nitro while the other is hydrogen; each of Y₁ and Y₂ may be the same or different, is phenyl which may be substituted by chlorine, fluorine or alkoxy; and m and n are integers from 0-4, a prodrug thereof, or a pharmaceutically acceptable salt thereof.

More preferably, the present invention contemplates a dihydropyridine-5-phosphonic acid cyclic propylene ester derivative of formula (I), wherein X₁ is hydrogen, X₂ is NO₂, m is 2, n is 1, Y₁ and Y₂ are phenyl.

Still more preferably, the present invention contemplates a dihydropyridine-5-phosphonic acid cyclic propylene ester derivative of formula (I), wherein X₁ is hydrogen, X₂ is NO₂, m is 2, n is 1, Y₁ and Y₂ are phenyl, R_{3,} R_{4,} R_{5,} R₆ are hydrogen, and R_{1,} R_{2,} R_{7,} R₈ are CH₃.

Compounds of formula (I) are useful to treat various cancers or pre-cancerous conditions, particularly those arising from neuronal, glial, epithelial, secretory, connective, muscle, or astrocyte cells. The cancers or pre-cancerous conditions that can be treated with compounds of formula (I) include, but are not limited to, cancers or pre-cancerous conditions arising in the colon, breast, ovary, uterus, prostate, liver, pancreas, central nervous system, skin, kidney, stomach, esophagus, lung and bronchus, lymphatic, hematopoetic, or the musculoskeletal system.

Compounds of formula (I) are further useful to treat epilepsy, autism, diabetic nephropathy, diabetic neuropathy, age adjusted macular degeneration, and scars, burns and keloids.

Examples of alkyl of one to six carbon atoms, inclusive, are methyl, ethyl, propyl, butyl, pentyl and hexyl and all isomeric forms and straight and branched chains thereof.

The term “alkoxy” is defined as a —OR′ radical, where R′ is an alkyl radical of 1-6 carbon atoms.

By “mammal” it is meant to refer to all mammals, including, for example, primates such as humans and monkeys. Examples of other mammals included herein are rabbits, dogs, cats, cattle, goats, sheep and horses. Preferably, the mammal is a female or male human.

The term “treating”, “treat” or “treatment” as used herein includes preventative (e.g., prophylactic) and palliative treatment.

The term “therapeutically effective amount” means an amount of a compound of the present invention that ameliorates, attenuates or eliminates a particular disease or condition or prevents or delays the onset of a particular disease or condition.

The phrase “compound(s) of the present invention” or “compound(s) of Formula I” or the like, shall at all times be understood to include all active forms of such compounds, including, for example, the free form thereof, e.g., the free acid or base form, and also, all prodrugs, polymorphs, hydrates, solvates, tautomers, and the like, and all pharmaceutically acceptable salts, unless specifically stated otherwise. It will also be appreciated that suitable active metabolites of such compounds are within the scope of the present invention.

By “pharmaceutically acceptable” it is meant the carrier, vehicle, diluent, excipient and/or salt must be compatible with the other ingredients of the formulation, and not deleterious to the recipient thereof.

The expression “prodrug” refers to compounds that are drug precursors which following administration, release the drug in vivo via some chemical or physiological process (e.g., a prodrug on being brought to the physiological pH or through enzyme action) is converted to the desired drug form.

The expression “pre-cancerous condition” refers to a growth that is not malignant but is likely to become so if not treated. A “pre-cancerous condition” is also known as “pre-malignant condition” by one of ordinary skill in the art.

The expressions “condition” or “conditions” refer to an injury, ailment or disease such as epilepsy, autism, diabetic nephropathy, diabetic neuropathy, age adjusted macular degeneration, scars, burns and keloids.

Compounds of the present invention are readily available in the commercial market or can be routinely prepared via methods well-known to one skilled in the art as described in U.S. Pat. No. 4,885,284, and thereby are economically affordable. The pharmaceutical activity of the compounds, prodrugs and pharmaceutically acceptable salts of the present invention are demonstrated by one or more of the assays described in U.S. Pat. No. 4,885,284 or other experimental protocols known to one skilled in the art. Biological studies show that compounds of the present invention inhibit the proliferation of cancer cells in vitro as well as in various experimental animal models of prostate, breast, and colon cancers. Additional studies demonstrate cytostatic effects of compounds of the present invention to lung, ovary, pancreas, and other cancerous tissues. It is also observed that cancer cells treated with compounds of the present invention do not develop resistance to those compounds.

Any of the compounds and prodrugs of the present invention can be synthesized as pharmaceutically acceptable salts for incorporation into various pharmaceutical compositions. As used herein, pharmaceutically acceptable salts include, but are not limited to, hydrochloric, hydrobromic, hydroiodic, hydrofluoric, sulfuric, sulfonic, citric, camphoric, maleic, acetic, lactic, nicotinic, nitric, succinic, phosphoric, malonic, malic, salicyclic, phenylacetic, stearic, palmitic, pyridine, ammonium, piperazine, diethylamine, nicotinamide, formic, fumaric, urea, sodium, potassium, calcium, magnesium, zinc, lithium, cinnamic, methylamino, methanesulfonic, picric, p-toluenesulfonic, naphthalenesulfonic, tartaric, triethylamino, dimethylamino, and tris(hydroxymethyl)aminomethane. Additional pharmaceutically acceptable salts would be apparent to one of ordinary skill in the art. Where more than one basic moiety exists, the expression includes multiple salts (e.g., di-salt).

Also, the compounds of the present invention, and any prodrugs and pharmaceutically acceptable salts thereof, can exist in several tautomeric forms, including the enol form, the imine form and mixtures thereof. All such tautomeric forms are included within the scope of the present invention.

In another embodiment, the present invention also provides pharmaceutical compositions comprising a therapeutically effective amount of a compound of formula (I), a prodrug of said compound or a pharmaceutically acceptable salt of said compound or prodrug; and a pharmaceutically acceptable carrier, vehicle or diluent.

The pharmaceutical compositions and compounds of the present invention, including prodrugs and pharmaceutically acceptable salts thereof, will generally be administered daily in the form of a dosage unit (e.g., tablet, capsule, etc.) at a therapeutically effective amount of such compound, prodrug or salt thereof from about 100 mg to about 10 g per day, more particularly from about 500 mg to about 3 g per day. As recognized by those skilled in the art, the particular quantity of pharmaceutical composition according to the present invention administered to a patient will depend upon a number of factors, including, without limitation, the activity desired, the condition of the patient (such as body weight, severity of the illness, and etc.), and tolerance for the compound.

The pharmaceutically acceptable carrier, vehicle or diluent includes, but is not limited to, any excepient that is generally recognized as safe by the U.S. Food and Drug Administration.

The pharmaceutical compositions and compounds of the present invention, including prodrugs and pharmaceutically acceptable salts thereof, can be administered through various routes including parenteral, intravenous, intramuscular, intraperitoneal, intrathecal, suppository, transdermal, topical, or oral. Oral administration of the pharmaceutical compositions and compounds of the present invention is most preferred.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds, isomers, prodrugs and pharmaceutically acceptable salts thereof of this invention can be combined with various sweetening agents, flavoring agents, coloring agents, emulsifying agents and/or suspending agents, as well as such diluents as water, ethanol, propylene glycol, glycerin and various like combinations thereof.

Due to their ease of administration, tablets and capsules represent the most advantageous oral dosage form for the pharmaceutical compositions of the present invention.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g., topical) administration, dilute sterile, aqueous or partially aqueous solutions (usually in about 0.1% to 5% concentration), otherwise similar to the above parenteral solutions, are prepared.

For topical administration, the compounds, prodrugs and pharmaceutically acceptable salts thereof of the present invention may be formulated using bland, moisturizing bases, such as ointments or creams. Examples of suitable ointment bases are petrolatum, petrolatum plus volatile silicones, lanolin, and water in oil emulsions.

Methods of preparing various pharmaceutical compositions with a certain amount of the compound of the present invention, a prodrug or salt thereof, are known, or will be apparent in light of this disclosure, to those skilled in this art. For examples of methods of preparing pharmaceutical compositions, see Remington's Pharmaceutical Sciences, Mack Publishing Company, Easter, Pa., 19th Edition (1995).

The pharmaceutical compositions and compounds of the present invention, including prodrugs and pharmaceutically acceptable salts thereof, can be administered continuously, in divided doses, or in a single dose per day. Preferably, the pharmaceutical compositions and compounds of the present invention are administered in divided daily doses.

The pharmaceutical compositions and compounds of the present invention, including prodrugs and pharmaceutically acceptable salts thereof, can be administered with adjuvant, neo-adjuvant, or preventive intent. Since compounds of the present invention causes cancer cells to stop growing and do not directly kill cancer cells, it would be particularly advantageous to use compounds of the present invention when the proliferation of cancer cells is at early stage. Thus, the pharmaceutical compositions and compounds of the present invention for the treatment of cancer are preferably used in preventive or adjuvant roles.

The present invention also provides a method for the treatment of cancer or pre-cancerous condition in a mammal, which comprises administering to the mammal a therapeutically effective amount of a compound of formula (I), a prodrug thereof, or a pharmaceutically acceptable salt of said compound or prodrug in combination with one or more antineoplastic agent.

The present invention further provides pharmaceutical combination compositions comprising a therapeutically effective amount of a combination of a compound of formula (I), a prodrug thereof, or a pharmaceutically acceptable salt of said compound or prodrug; and one or more antineoplastic agent.

It will be understood by those skilled in the art that the compounds, prodrugs and pharmaceutically acceptable salts thereof, including pharmaceutical compositions and formulations containing these compounds, prodrugs and salts can be used in a wide variety of combination therapies to treat cancers and pre-cancerous conditions described above. Thus, the compounds, prodrugs and pharmaceutically acceptable salts thereof of the present invention can be used in conjunction with other antineoplastic agents for the treatment of cancers and pre-cancerous conditions described herein.

Any known, commercially marketed antineoplastic agents or anticancer drugs may be used as the other antineoplastic agents in the combination aspect of this invention. Other suitable antineoplastic agents include, but not limit to, cytotoxic agents (such as alkylating agents, antimetabolites and cytotoxic antibiotics), and cytostatic agents (such as antiangiogenic agents).

In combination therapy treatment, both the compounds of this invention and the other antineoplastic agents are administered to mammals (e.g., humans, male or female) by the methods described hereinabove. As recognized by those skilled in the art, the therapeutically effective amounts of the compounds of this invention and the other antineoplastic agents to be administered to a patient in combination therapy treatment will depend upon a number of factors, including, without limitation, the biological activity desired, the condition of the patient, and tolerance for the compound. Further, the compounds of this invention and the other antineoplastic agents in combination therapy may be administered simultaneously, separately, or sequentially in the same or different dosage forms (e.g., oral and parenteral). The compounds of this invention and the other antineoplastic agents in combination therapy may also be administered at the same or different dosage intervals, or when titration of the individual components of the combination is desired by the prescribing physician.

1. A method for the treatment of cancer, a pre-cancerous condition or other conditions in a mammal, which comprises administering to the mammal a therapeutically effective amount of a compound of formula (I) 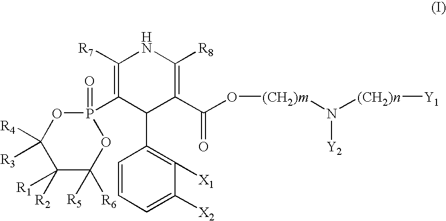
wherein each of R₁-R₈ are the same or different, are hydrogen or C₁-C₆ alkyl; one of X₁ and X₂ is nitro while the other is hydrogen; each of Y₁ and Y₂ may be the same or different, is phenyl which may be substituted by chlorine, fluorine or alkoxy; and m and n are integers from 0-4, a prodrug thereof, or a pharmaceutically acceptable salt thereof.

2. The method according to claim 1, wherein X₁ is hydrogen, X₂ is NO₂, m is 2, n is 1, Y₁ and Y₂ are phenyl.

3. The method according to claim 2, wherein R_{3,} R_{4,} R_{5,} R₆ are hydrogen, and R_{1,} R_{2,} R_{7,} R₈ are CH₃.

4. The method according to claim 1, wherein said other conditions are selected from the group consisting of epilepsy, autism, diabetic nephropathy, diabetic neuropathy, age adjusted macular degeneration, and scars, burns and keloids.

5. A method for the treatment of a cancer or a pre-cancerous condition in a mammal, which comprises administering to the mammal a therapeutically effective amount of a compound of formula (I) as described in claim 1 a prodrug thereof, or a pharmaceutically acceptable salt of said compound or prodrug in combination with one or more antineoplastic agent.

6. A pharmaceutical composition comprising a therapeutically effective amount of a compound of formula (I) as described in claim 1, a prodrug of said compound or a pharmaceutically acceptable salt of said compound or prodrug; and a pharmaceutically acceptable carrier, vehicle or diluent.

7. A pharmaceutical combination composition comprising a therapeutically effective amount of a combination of a compound of formula (I) as described in claim 1, a prodrug thereof, or a pharmaceutically acceptable salt of said compound or prodrug; and one or more antineoplastic agent.
